(19) United States
(12) Patent Application Publication
(10) Pub. No.: US 2024/0296907 A1
(43) Pub. Date: Sep. 5, 2024

(54) METHOD, APPARATUS AND DEVICE FOR ANALYZING GENOME METHYLATION SEQUENCING DATA, AND MEDIUM

(71) Applicants: Chengdu BOE Optoelectronics Technology Co., Ltd., Chengdu, Sichuan (CN); BOE TECHNOLOGY GROUP CO., LTD., Beijing (CN)

(72) Inventor: Yang Song, Beijing (CN)

(73) Assignees: Chengdu BOE Optoelectronics Technology Co., Ltd., Chengdu, Sichuan (CN); BOE TECHNOLOGY GROUP CO., LTD., Beijing (CN)

(21) Appl. No.: 18/025,544

(22) PCT Filed: Mar. 31, 2022
(86) PCT No.: PCT/CN2022/084386

§ 371 Date: Mar. 9, 2023

(51) Int. Cl. G16B 30/00

(52) U.S. Cl. CPC G16B 30/00

(57) ABSTRACT

A method for analyzing genome methylation sequencing data includes: acquiring a genome methylation sequencing sequence to be detected and a reference genome sequence; aligning the genome methylation sequencing sequence to the reference genome sequence so as to obtain an alignment result; constructing a window, and moving the window from a first end of the alignment result to a second end of the alignment result successively, and counting a methylation index of a part of the alignment result covered by the window at a different position during each movement, a step size of each movement of the window being smaller than a length of the window; and analyzing a counted methylation index of the part of the alignment result covered by a window at a respective different position, and outputting a comprehensive methylation evaluation result of the genome methylation sequencing sequence.

### TECHNICAL FIELD

The disclosure belongs to the technical field of gene detection, and more particularly, to a method, an apparatus and a device for analyzing genome methylation sequencing data, and a medium.

### BACKGROUND

DNA (Deoxyribo Nucleic Acid) methylation is an epigenetic modification without changing a DNA sequence, that is, a process of adding methyl to a 5′ carbon of cytosine. The DNA methylation in a human body generally occurs at CpG nucleotide site, which can regulate expression of coding genes. A methylation pattern of the genome may be obtained by using high-throughput sequencing technologies. It has shown in studies that DNA methylation patterns play an important role in regulating individual growth, development, gene expression patterns and genome stability, and abnormal DNA methylation is closely related to occurrence and development of tumors and cell canceration. It is a trend in the field of disease surveillance to identify methylation patterns of individuals through methylation sequencing so as to obtain personalized disease assessment.

### SUMMARY

A method, an apparatus and a device for analyzing genome methylation sequencing data and a medium are provided in the present disclosure.

A method for detecting genome methylation sequencing data is provided in some embodiments of the present disclosure, which includes:
acquiring a genome methylation sequencing sequence to be detected and a reference genome sequence;
aligning the genome methylation sequencing sequence to the reference genome sequence so as to obtain an alignment result;
constructing a window, and moving the window from a first end of the alignment result to a second end of the alignment result successively, and counting a methylation index of a part of the alignment result covered by the window at a different position during each movement, a step size of each movement of the window being smaller than a length of the window; and
analyzing a counted methylation index of the part of the alignment result covered by a window at a respective different position, and outputting a comprehensive methylation evaluation result of the genome methylation sequencing sequence.

Optionally, when the comprehensive methylation evaluation result is the regional methylation level value of a target area in the alignment result, the methylation index including a total number of methylated bases at methylation sites for the alignment result covered by the window in the target area, and a total number of bases at the methylation sites;
the step of analyzing the counted methylation index of the part of the alignment result covered by the window at the respective different position and outputting the comprehensive methylation evaluation result of the genome methylation sequencing sequence includes:
calculating a regional window methylation level value corresponding to each window at the different position in the target area, according to a ratio of the total number of the methylated bases at the methylation sites to the total number of bases at the methylation sites included in the alignment result covered by a window at a respective different position in the target area; and
analyzing the regional window methylation level value corresponding to each window at the different position in the target region to obtain the regional methylation level value of the target region in the genome methylation sequencing sequence.

Optionally, analyzing the regional window methylation level value corresponding to each window at the different position in the target region to obtain the regional methylation level value of the target region in the genome methylation sequencing sequence includes:
averaging the regional window methylation level value corresponding to each window at the different position in the target region, and calculating the regional methylation level value of the target region in the genome methylation sequencing sequence according to an average of the regional window methylation level value.

Optionally, when the comprehensive methylation evaluation result is a site methylation level value of a target site, the methylation index includes a total number of bases at the methylation sites in the alignment result covered by a window covering the target site and a number of methylated bases at the target site;
the step of analyzing the counted methylation index of the part of the alignment result covered by a window at a respective different position and outputting a comprehensive methylation evaluation result of the genome methylation sequencing sequence includes:
calculating a site-window methylation level value corresponding to a window covering a respective different position of the target site according to a ratio of the number of the methylated bases at the target site to the total number of the bases at the methylation sites in the alignment result covered by different windows covering the target site; and
analyzing the site-window methylation level value corresponding to the window covering the respective different position of the target site to obtain the site methylation level value of the target site.

Optionally, analyzing the site-window methylation level value corresponding to the window covering the respective different position of the target site to obtain the site methylation level value of the target site includes:
averaging the site-window methylation level value corresponding to the window covering the respective different position of the target site, and calculating the site methylation level value of the target site in the genome methylation sequencing sequence according to an average of the site-window methylation level value.

Optionally, sliding the window from the first end of the alignment result to the second end of the alignment result successively and counting the methylation index of the part of the alignment result covered by the window at the different position during each movement includes:
sliding the window from the first end of the alignment result to the second end of the alignment result by a preset length, counting a methylation index of a covered alignment result before a first sliding, and counting a methylation index of the alignment result covered by the window after each sliding, wherein a step size of each moving of the window is smaller than the length of the window.

Optionally, aligning the genome methylation sequencing sequence to the reference genome sequence so as to obtain the alignment result includes:
segmenting the reference genome sequence to obtain a plurality of reference genomic fragment sequences; and
aligning each of the reference genomic fragment sequences to the genome methylation sequencing sequence in terms of negative and positive strands so as to obtain the alignment result.

Optionally, segmenting the reference genome sequence to obtain the plurality of reference genomic fragment sequences includes:
segmenting the reference genome sequence by a chromosome unit to obtain a plurality of reference chromosome genomic sequences; and
segmenting each of the reference chromosome genomic sequences by a preset length to obtain the plurality of reference genomic fragment sequences.

Optionally, the reference genome sequence includes a first converted reference genome sequence and a second converted reference genome sequence, and the genome methylation sequencing sequence at least includes a first amplified methylated genomic sequence and a second amplified methylated genomic sequence;
the step of aligning each of the reference genomic fragment sequences to the genome methylation sequencing sequence in terms of negative and positive strands so as to obtain the alignment result includes:
performing base conversion on the first amplified methylated genomic sequence to at least obtain a third amplified methylated genomic sequence and obtain a fourth amplified methylated genomic sequence, performing base conversion on the second amplified methylated genomic sequence to at least obtain a fifth amplified methylated genomic sequence and a sixth amplified methylated genomic sequence;
aligning the first converted reference genome sequence and the second converted reference genome sequence to the third amplified methylated genomic sequence, the fourth amplified methylated genomic sequence, the fifth amplified methylated genomic sequence and the sixth amplified methylated genomic sequence respectively; and
taking a mother sequence of an amplified methylated genomic sequence which is aligned to be identical to the first converted reference genome sequence as the positive strand, and taking a mother sequence of an amplified methylated genomic sequence which is aligned to be identical to the second converted reference genome sequence as the negative strand.

Optionally, the step of performing base conversion on the first amplified methylated genomic sequence to at least obtain the third amplified methylated genomic sequence and obtain the fourth amplified methylated genomic sequence, and performing base conversion on the second amplified methylated genomic sequence to at least obtain the fifth amplified methylated genomic sequence and the sixth amplified methylated genomic sequence includes:
performing base conversion from C to T on the first amplified methylated genomic sequence to obtain the third amplified methylated genomic sequence, performing base conversion from G to A on the first amplified methylated genomic sequence to obtain the fourth amplified methylated genomic sequence; and
performing the base conversion from C to T on the second amplified methylated genomic sequence to obtain the fifth amplified methylated genomic sequence, and performing the base conversion from G to A on the second amplified methylated genomic sequence to obtain the sixth amplified methylated genomic sequence.

Optionally, after methylated genomic sequencing to acquire the genome methylation sequencing sequence to be detected and obtaining the reference genome sequence by downloading from a database, the method further includes:
pruning a sequence of a target type in the acquired original gene sequencing sequences, wherein the sequence of the target type includes at least one of: an adapter sequence, a sequence overlapping with the adapter sequence by more than a preset number of bases, a terminal sequence with a mass value lower than a mass value threshold, and after completely pruning, discarding a sequence with a length lower than a length threshold to obtain pruned original gene sequencing sequences, and then filtering the pruned original gene sequencing sequences; and
when the pruned filtered original gene sequencing sequences do not meet target requirements, continuously performing filtering on the pruned original gene sequencing sequences until the filtered pruned-original gene sequencing sequences meet the target requirements, and taking the filtered pruned-original gene sequencing sequences as the genome methylation sequencing sequence to be detected, wherein the target requirements include at least one of a base quality requirement, a base ratio requirement, an average sequence GC distribution requirement, a N content distribution requirement, a sequence length requirement, a repeated sequence requirement and an adapter sequence requirement.

An apparatus for analyzing genome methylation sequencing data is provided in some embodiments of the present disclosure, which includes:
an acquisition module configured to acquire a genome methylation sequencing sequence to be detected and a reference genome sequence;
an alignment module configured to align the genome methylation sequencing sequence to the reference genome sequence to obtain an alignment result;
a counting module configured to construct a window, and move the window from a first end of the alignment result to a second end of the alignment result successively, and count a methylation index of a part of the alignment result covered by the window at a different position during each movement, a step size of each movement of the window is smaller than a length of the window; and
an evaluation module configured to analyze the counted methylation index of the part of the alignment result covered by a window at a respective different position and output a comprehensive methylation evaluation result of the genome methylation sequencing sequence.

Optionally, when the comprehensive methylation evaluation result is the regional methylation level value of a target area in the alignment result, the methylation index including a total number of methylated bases at methylation sites for the alignment result covered by the window in the target area, and a total number of bases at the methylation sites;
the evaluation module is further configured for:
calculating a regional window methylation level value corresponding to each window at the different position in the target area, according to a ratio of the total number of the methylated bases at the methylation sites to the total number of bases at the methylation sites included in the alignment result covered by a window at a respective different position in the target area; and
analyzing the regional window methylation level value corresponding to each window at the different position in the target region to obtain the regional methylation level value of the target region in the genome methylation sequencing sequence.

Optionally, the evaluation module is further configured for:
averaging the regional window methylation level value corresponding to each window at the different position in the target region, and calculating the regional methylation level value of the target region in the genome methylation sequencing sequence according to an average of the regional window methylation level value.

Optionally, when the comprehensive methylation evaluation result is a site methylation level value of a target site, the methylation index includes a total number of bases at the methylation sites in the alignment result covered by a window covering the target site and a number of methylated bases at the target site;
the evaluation module is further configured for:
calculating a site-window methylation level value corresponding to a window covering a respective different position of the target site according to a ratio of the number of the methylated bases at the target site to the total number of the bases at the methylation sites in the alignment result covered by different windows covering the target site; and
analyzing the site-window methylation level value corresponding to the window covering the respective different position of the target site to obtain the site methylation level value of the target site.

Optionally, the evaluation module is further configured for:
averaging the site-window methylation level value corresponding to the window covering the respective different position of the target site, and calculating the site methylation level value of the target site in the genome methylation sequencing sequence according to an average of the site-window methylation level value.

Optionally, the counting module is further configured for:
sliding the window from the first end of the alignment result to the second end of the alignment result by a preset length, counting a methylation index of a covered alignment result before a first sliding, and counting a methylation index of the alignment result covered by the window after each sliding, wherein a step size of each moving of the window is smaller than the length of the window.

Optionally, the alignment module is further configured for:
segmenting the reference genome sequence to obtain a plurality of reference genomic fragment sequences; and
aligning each of the reference genomic fragment sequences to the genome methylation sequencing sequence in terms of negative and positive strands so as to obtain the alignment result.

Optionally, the alignment module is further configured for:
segmenting the reference genome sequence by a chromosome unit to obtain a plurality of reference chromosome genomic sequences; and
segmenting each of the reference chromosome genomic sequences by a preset length to obtain the plurality of reference genomic fragment sequences.

Optionally, the reference genome sequence includes a first converted reference genome sequence and a second converted reference genome sequence, and the genome methylation sequencing sequence at least includes a first amplified methylated genomic sequence and a second amplified methylated genomic sequence;
the alignment module is further configured for:
performing base conversion on the first amplified methylated genomic sequence to at least obtain a third amplified methylated genomic sequence and obtain a fourth amplified methylated genomic sequence, performing base conversion on the second amplified methylated genomic sequence to at least obtain a fifth amplified methylated genomic sequence and a sixth amplified methylated genomic sequence;
aligning the first converted reference genome sequence and the second converted reference genome sequence to the third amplified methylated genomic sequence, the fourth amplified methylated genomic sequence, the fifth amplified methylated genomic sequence and the sixth amplified methylated genomic sequence respectively; and
taking a mother sequence of an amplified methylated genomic sequence which is aligned to be identical to the first converted reference genome sequence as the positive strand, and taking a mother sequence of an amplified methylated genomic sequence which is aligned to be identical to the second converted reference genome sequence as the negative strand.

Optionally, the alignment module is further configured for:
performing base conversion from C to T on the first amplified methylated genomic sequence to obtain the third amplified methylated genomic sequence, performing base conversion from G to A on the first amplified methylated genomic sequence to obtain the fourth amplified methylated genomic sequence; and
performing the base conversion from C to T on the second amplified methylated genomic sequence to obtain the fifth amplified methylated genomic sequence, and performing the base conversion from G to A on the second amplified methylated genomic sequence to obtain the sixth amplified methylated genomic sequence.

Optionally, the acquisition module is further configured for:
pruning a sequence of a target type in the acquired original gene sequencing sequences, wherein the sequence of the target type includes at least one of: an adapter sequence, a sequence overlapping with the adapter sequence by more than a preset number of bases, a terminal sequence with a mass value lower than a mass value threshold, and after completely pruning, discarding a sequence with a length lower than a length threshold to obtain pruned original gene sequencing sequences, and then filtering the pruned original gene sequencing sequences; and
when the pruned original gene sequencing sequences do not meet target requirements, continuously performing filtering on the pruned original gene sequencing sequences until the filtered pruned-original gene sequencing sequences meet the target requirements, and taking the filtered pruned-original gene sequencing sequences as the genome methylation sequencing sequence to be detected, wherein the target requirements include at least one of a base quality requirement, a base ratio requirement, an average sequence GC distribution requirement, a N content distribution requirement, a sequence length requirement, a repeated sequence requirement and an adapter sequence requirement.

A computing and processing device is provided in some embodiments of the present disclosure, which includes:
a memory with a computer-readable code stored therein;
one or more processors, the computing and processing device executing the method for analyzing the genome methylation sequencing data stated above when the computer-readable code is executed by the one or more processors.

A computer program, including a computer-readable code is provided in some embodiments of the present disclosure, which, when executed on a computing and processing device, causes the computing and processing device to execute the method for analyzing the genome methylation sequencing data stated above.

A non-transient computer-readable medium with a computer program of the method for analyzing the genome methylation sequencing data stated above stored therein.

The above description is merely a summary of the technical solutions of the present disclosure. In order to more clearly know the elements of the present disclosure to enable the implementation according to the contents of the description, and in order to make the above and other purposes, features and advantages of the present disclosure more apparent and understandable, the particular embodiments of the present disclosure are provided below.

### BRIEF DESCRIPTION OF THE DRAWINGS

In order to explain embodiments of the present disclosure or the technical scheme in the prior art more clearly, the drawings required in the description of the embodiments or the prior art will be briefly introduced below; obviously, the drawings in the following description are some embodiments of the present disclosure, and other drawings may be obtained according to these drawings by those of ordinary skill in the art without paying creative labor.

FIG. 1 schematically shows a flow chart of a method for analyzing genome methylation sequencing data according to some embodiments of the present disclosure;

FIG. 2 schematically shows a flow chart of another method for analyzing genome methylation sequencing data according to some embodiments of the present disclosure;

FIG. 3 schematically shows a schematic diagram of the principle of another method for analyzing genome methylation sequencing data according to some embodiments of the present disclosure;

FIG. 4 schematically shows another flow chart of another method for analyzing genome methylation sequencing data according to some embodiments of the present disclosure;

FIG. 5 schematically shows another schematic diagram of the principle of another method for analyzing genome methylation sequencing data according to some embodiments of the present disclosure;

FIG. 6 schematically shows another flow chart of another method for analyzing genome methylation sequencing data according to some embodiments of the present disclosure;

FIG. 7 schematically shows another schematic diagram of the principle of another method for analyzing genome methylation sequencing data according to some embodiments of the present disclosure;

FIG. 8 schematically shows another flow chart of another method for analyzing genome methylation sequencing data according to some embodiments of the present disclosure;

FIG. 9 schematically shows another schematic diagram of the principle of another method for analyzing genome methylation sequencing data according to some embodiments of the present disclosure;

FIG. 10 schematically shows another schematic diagram of the principle of another method for analyzing genome methylation sequencing data according to some embodiments of the present disclosure;

FIG. 11 schematically shows another flow chart of another method for analyzing genome methylation sequencing data according to some embodiments of the present disclosure;

FIG. 12 schematically shows a flow chart of yet another method for analyzing genome methylation sequencing data according to some embodiments of the present disclosure;

FIG. 13 schematically shows effect schematic diagram of yet another method for analyzing genome methylation sequencing data according to some embodiments of the present disclosure;

FIG. 14 schematically shows another effect schematic diagram of yet another method for analyzing genome methylation sequencing data according to some embodiments of the present disclosure;

FIG. 15 schematically shows another effect schematic diagram of yet another method for analyzing genome methylation sequencing data according to some embodiments of the present disclosure;

FIG. 16 schematically shows another effect schematic diagram of yet another method for analyzing genome methylation sequencing data according to some embodiments of the present disclosure;

FIG. 17 schematically shows a schematic structural diagram of an apparatus for analyzing genome methylation sequencing data according to some embodiments of the present disclosure;

FIG. 18 schematically shows a block diagram of a computing and processing device for executing the method according to some embodiments of the present disclosure; and

FIG. 19 schematically shows a storage unit for holding or carrying program codes for implementing the method according to some embodiments of the present disclosure.

### DETAILED DESCRIPTION OF THE EMBODIMENTS

In order to make the objects, the technical solutions, and the advantages of the present disclosure clearer, the technical solutions of the embodiments of the present application will be clearly and completely described below with reference to the drawings of the embodiments of the present application. Apparently, the described embodiments are merely certain embodiments of the present application, rather than all of the embodiments. All of the other embodiments that a person skilled in the art obtains on the basis of the embodiments of the present application without paying creative work fall within the protection scope of the present application.

In related art, a genome methylation sequencing sequence is usually obtained by high-throughput sequencing technologies, and then the whole genome methylation sequencing sequence is aligned to a pre-prepared reference genome sequence, so as to identify a methylation level of the genome methylation sequencing sequence according to the alignment result. However, due to the influence of the alignment process needing a whole alignment, the alignment process takes a long time, with a low alignment efficiency. Moreover, due to errors in upstream experiment and sequencing processes, such as incomplete methylation conversion at methylation sites in the experiment or a low sequencing resolution, a methylation level at some sites or regions cannot be identified or identified accurately.

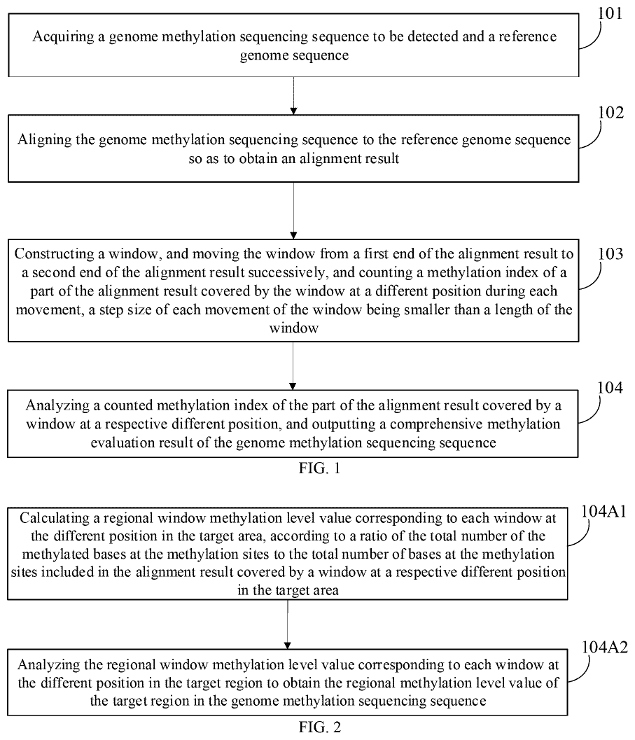

FIG. 1 schematically shows a flow chart of a method for analyzing genome methylation sequencing data according to the present disclosure, which includes following steps S101 to S104.

In the step 101, a genome methylation sequencing sequence to be detected and a reference genome sequence is acquired.

It should be noted that the genome methylation sequencing sequence to be detected refers to a human genome sequence obtained by sequencing the methylated genome obtained in the upstream experiment by high-throughput sequencing technologies, and the reference genome sequence is a high-quality human genome sequence obtained by downloading from a global gene database.

In the embodiment of the present disclosure, after the genome methylation sequencing sequence is acquired, the genome methylation sequencing sequence may be further subjected to quality screening and data filtering, so as to improve the quality of the genome methylation sequencing sequence participating in subsequent identification and ensure the accuracy of methylation index identification. For example, a low-quality gene sequencing sequence may be filtered according to a base ratio and a base distribution of the genome methylation sequencing sequence, or may be pretreated by means of de-duplication, removal of incomplete fragments, etc. A specific pretreatment method may be set according to actual needs, which is not limited herein.

In the step 102, the genome methylation sequencing sequence is aligned to the reference genome sequence so as to obtain an alignment result.

In the embodiment of the present disclosure, the genome methylation sequencing sequence is aligned to the reference genome sequence so as to obtain the alignment result of the genome methylation sequencing sequence. In the alignment process, DNA (Deoxyribo Nucleic Acid) after methylation conversion is subjected to PCR (Polymerase Chain Reaction) amplification in the upstream experiment, and in order to ensure adequacy of the alignment result, it is necessary to further perform base conversion on the methylated sequencing sequence and the reference genome sequence, so that various possible combination sequences may be aligned in the alignment process. Based on the alignment result, a number of methylated bases at different sites or regions in the genome methylation sequencing sequence may be calculated for use in a subsequent identification process of a methylation level.

Further, due to preference of PCR amplification in the upstream experiment, distribution of reads (sequence fragments) in some areas of the methylated gene sequencing sequence may be uneven, so the sequence may be sorted by alignment positions with the alignment result in a bam file format, and de-duplication may be performed on sequences with a same label, errors caused by the preference of the PCR amplification may be removed.

In the step 103, a window is constructed, and the window is moved from a first end of the alignment result to a second end of the alignment result successively, and a methylation index of a part of the alignment result covered by the window at a different position during each movement is counted, a step size of each movement of the window is smaller than a length of the window.

In the embodiment of the present disclosure, the window is an object for defining a value interval of the data, and a value interval of the data to be processed may be defined by covering a specific data interval by a value positional range of the window. In the present disclosure, the alignment result is a series of continuous data, and in order to analyze and count some segments of or part of the alignment result, the alignment result within a range of values covered by the window may be located through the window covering a part of the alignment result. Specifically, the window may be moved by sliding, jumping, etc. over the alignment result, so as to cover different value intervals in the alignment result.

In the embodiment of the present disclosure, it is considered that there may be errors in the DNA experiment process and sequencing process, for example, the methylation sites are not completely methylated conversion in the experiment process, or the resolution is too low in a methylation sequencing process, which may lead to inaccurate methylation levels of some sites or regions, and these errors may affect accuracy of a methylation evaluation result of sites or regions. Therefore, in the embodiment of the present disclosure, a window-type identification method is adopted to reduce negative effects caused by upstream experimental errors and sequencing errors.

Specifically, in the embodiment of the present disclosure, the window covering a part of the alignment result is moved from one end of an area to be detected to the other end of an area to be detected in the alignment result successively, with a same sliding step size and a same length of the window for each time, and the sliding step size should be smaller than the length of the window each time, so as to ensure that areas covered by the window at different positions have overlapping parts, and in sliding, the methylation index of a different area covered by the window in each sliding over the alignment result may be counted. Therefore, methylation indexes in the alignment result may be divided into a plurality of different regions, with some overlapping regions, and the methylation indexes corresponding to the windows at different positions with multiple coverage may be obtained.

In the step 104, the counted methylation index of the part of the alignment result covered by the window at the respective different position may be analyzed, and a comprehensive methylation evaluation result of the genome methylation sequencing sequence may be output.

In the embodiment of the present disclosure, the methylation indexes of the alignment result covered by the window at different positions are analyzed as the comprehensive methylation evaluation result. A way of integration may be weighted fitting, averaging, etc., which may be specifically set according to actual needs and is not limited herein.

According to the embodiment of the present disclosure, the comprehensive methylation evaluation result obtained by integrating counted methylation indexes by different windows with multiple coverage can summarize methylation indexes of the alignment result covered by multiple windows covering specific areas or characteristic sites, so that influence of local errors in the sequencing sequence on the accuracy of the methylation evaluation result of a whole area or site is reduced as much as possible, and accuracy of the methylation evaluation result of the methylated sequencing sequence may be improved.

In some embodiments of the present disclosure, for an identification process for regional methylation level values and site methylation level values, specific evaluation methods are shown in the present disclosure in the following. The two evaluation methods of the methylation level values may be used in parallel or independently in practical applications, and an order may be set according to actual needs when combined with each other, and the order does not affect accuracy of an obtained result.

### 1) Identification Method of the Regional Methylation Level:

Optionally, when the comprehensive methylation evaluation result is the regional methylation level value of a target area in the alignment result, the methylation index includes: a total number of methylated bases at methylation sites for the alignment result covered by the window in the target area, and a total number of bases at the methylation sites;

Referring to FIG. 2, the step 104 includes following step 104A1 and 104A2.

In step 104A1, a regional window methylation level value corresponding to each window at the different position in the target area may be calculated according to a ratio of the total number of the methylated bases at the methylation sites to the total number of bases at the methylation sites included in the alignment result covered by a window at a respective different position in the target area.

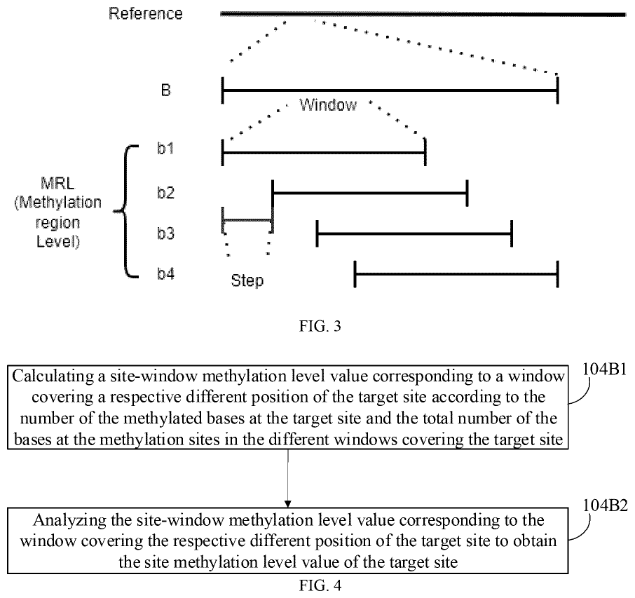

In the embodiment of the present disclosure, referring to FIG. 3, where Reference represents a reference genome sequence, Window represents a window, B represents an alignment region in the alignment result, b1 to b4 respectively represent 4 windows at different positions, Step represents a step size between adjacent windows, and MRL (Methylation region level) represents a regional methylation level value. Of course, this is only an exemplary description. A number of the windows may be any number greater than or equal to 2, and a sliding step size may also be any length less than a length of the window.

Referring to FIG. 3, the window starts from a left end starting position of the target area B at which time the window is marked as b1, and slides to a right end of the target area B in an order of b2, b3 and b4 according to Step. Each time when the window is located at a different position, that is, at an area covered by b1 to b4, the total number of methylated bases at the methylation sites and the total number of bases at the methylation sites in the covered area are counted.

In step 104A2, the regional window methylation level value corresponding to each window at the different position in the target region is analyzed to obtain the regional methylation level value of the target region in the genome methylation sequencing sequence.

In the embodiment of the present disclosure, for identification of the regional methylation level value, the regional methylation level value of the target region may be obtained by analyzing regional window methylation level values counted for a plurality of different windows covering only a part of the target region, so that influence of local errors in the sequencing sequence on the accuracy of the methylation level of the whole region may be reduced as much as possible, and the accuracy of the methylation evaluation result of the methylated sequencing sequence is improved. A specific way of integration may be weighted fitting, averaging, etc., which may be specifically set according to actual needs and is not limited herein.

Optionally, the step 104A2 includes averaging the regional window methylation level value corresponding to each window at the different position in the target region, and calculating the regional methylation level value of the target region in the genome methylation sequencing sequence according to an average of the regional window methylation level value.

In some embodiments of the present disclosure, the methylation level value of the window in the target area is firstly calculated by a following formula (1):

$$Mbi = Mi / ( Mi + UMi ) \qquad ( 1 )$$

wherein Mbi represents a regional window methylation level value of the i-th window, Mi represents a total number of methylated bases at methylation sites covered by the i-th window, UMi represents a total number of unmethylated bases at the methylation sites covered by the i-th window, and a sum of Mi and UMi represents a total number of bases at the methylation sites covered by the i-th window.

Then the regional window methylation level values for all windows in different positions in the target area are analyzed by a following formula (2):

$$M R L = ( {\sum }_{i = 1}^{n} Mbi ) / C \qquad ( 2 )$$

wherein MRL represents the regional methylation level value of the target area, i is a corner marker of a window, and C represents a number of windows obtained after the calculated target area is obtained.

### 2) Identification Method of Site Methylation Level:

Optionally, when the comprehensive methylation evaluation result is the site methylation level value of a target site, the methylation index includes a total number of bases at the methylation sites in a window covering the target site and a number of methylated bases at the target site. Referring to FIG. 4, the step 104 includes following step 104B1 and 104B2.

In step 104B1, a site-window methylation level value corresponding to a window covering a respective different position of the target site is calculated according to a ratio of the number of the methylated bases at the target site to the total number of the bases at the methylation sites in different windows covering the target site.

In the embodiment of the present disclosure, referring to FIG. 5, where Reference represents a reference genome sequence, Window represents a window, B represents an alignment region in the alignment result, b1 to b4 respectively represent 4 windows at different positions, Step represents a step size between adjacent windows, and M1 represents a target site (all of points marked by diamonds are target sites). Of course, this is only an exemplary description. A number of the windows may be any number greater than or equal to 2, and a sliding step size may also be any length less than a length of the window.

Referring to FIG. 5, the window starts from a left end starting position of the target area B at which time the window is marked as b1, and slides to a right end of the target area B in an order of b2, b3 and b4 according to Step. It may be seen that the number of target sites in an area covered by the window is different every time when the window is located at a different position, that is, at the area covered by b1 to b4. For example, a number of windows covering a first target site (counted from left to right) is 2, a number of windows covering a second target site is 3, and a number of windows covering a third target site is 4. A number of methylated bases covering a specific target site and a total number of bases at the methylation sites in different windows covering the target site are counted.

In the step 104B2, the site-window methylation level value corresponding to the window covering the respective different position of the target site is analyzed to obtain the site methylation level value of the target site.

In the embodiment of the present disclosure, for identification of the site methylation level value, the site methylation level value of the target site may be obtained by analyzing site-window methylation level values counted by a plurality of different windows covering the target site, so that the influence of local errors in the sequencing sequence on accuracy of a methylation evaluation result of the whole site may be reduced as much as possible, and the accuracy of the methylation evaluation result of the methylated sequencing sequence is improved. A specific way of integration may be weighted fitting, averaging, etc., which may be specifically set according to actual needs and is not limited herein.

Optionally, the step 104B2 includes averaging the site-window methylation level value corresponding to the window covering the respective different position of the target site, and calculating the site methylation level value of the target site in the genome methylation sequencing sequence according to an average of the site-window methylation level value.

In the embodiment of the present disclosure, a site-window methylation level value of each window covering the target site is firstly calculated by a following formula (3):

$$Msi = Ms / ( Mi + UMi ) \qquad ( 3 )$$

wherein Msi represents the site-window methylation level value of the i-th window covering the target site, Ms represents the number of methylated bases at the target site, Mi represents a total number of methylated bases of methylated sites covered by the i-th window, UMi represents a total number of unmethylated bases of the methylated sites covered by the i-th window, and a sum of Mi and UMi represents a total number of base at the methylation sites covered by the i-th window.

Then, all site-window methylation level values of respective windows covering the target site are analyzed with a following formula (4) so as to obtain the site methylation level value of the target site:

$$M S L = ( {\sum }_{i = 1}^{n} Msi ) / c \qquad ( 4 )$$

wherein MSL (Methylation site level) represents the site methylation level value of the target site, i represents a corner marker of the window, and c represents a number of windows repeatedly covering the target site.

Optionally, the step 103 includes sliding the window from the first end of the alignment result to the second end of the alignment result by a preset length, counting a methylation index of the covered alignment result before a first sliding, and counting a methylation index of the alignment result covered by the window after each sliding. A step size for each moving of the window is smaller than the length of the window.

In some embodiments of the present disclosure, the length of the window partially covering the alignment result is set as a, and a length of the alignment result is expressed as L, so that a is less than L, and the sliding step size is preset as s, so that s is less than a, and both a and s are negatively correlated with a number of times the window slides. It is worth noting that, within a certain range, the smaller the length of the window, the smaller the sliding step size is and the greater the number of sliding times is, and thus the more repeated parts of a respective window there are, the larger amount of data it is obtained, and the more sufficient the calculated methylation index is, thus making a subsequent calculated comprehensive methylation evaluation result more accurate. On the contrary, if the length of the window is larger and the sliding step size is larger, the amount of data of the obtained methylation index may be smaller, time consumption of data counting may be less, and efficiency of methylation evaluation may be improved. Of course, the length of the window and the step size for each sliding may be set according to actual needs, the length may be set to be applied in the embodiments of the present disclosure when the obtained window covers the alignment result multiple times, which is not limited herein.

Further, if the preset step size for each window sliding may be greater than or equal to half the length of the window, data of the alignment result covered multiple times may be guaranteed to be continuous, so that the alignment result may be covered multiple times as far as possible, and thus an evaluation result of a methylation level obtained in the subsequent evaluation process are more accurate.

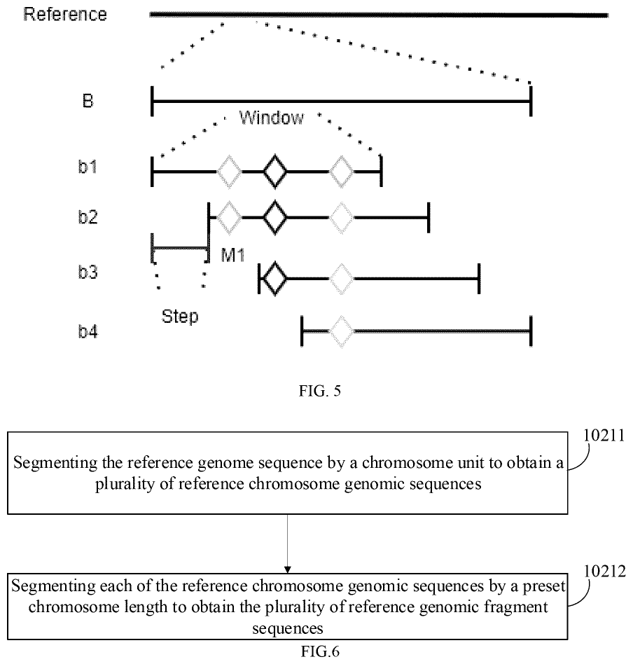

Optionally, referring to FIG. 6, the step 102 includes following steps 1021 and 1022.

In step 1021, the reference genome sequence is segmented to obtain a plurality of reference genomic fragment sequences.

In the embodiment of the present disclosure, the reference genome sequence may be segmented according to a specific scale or a specific unit, such as a chromosome unit or the like, to obtain a plurality of reference chromosome genomic sequences.

In step 1022, each of the reference genomic fragment sequences is aligned to the genome methylation sequencing sequence in terms of negative and positive strands so as to obtain the alignment result.

In some embodiments of the present disclosure, compared with a way of aligning all of the methylated sequencing sequences to the whole reference genome sequence in the related art, alignment time consumption may be significantly reduced thereby improving an alignment throughput and speed after sequencing because the methylated sequencing sequence is aligned to a plurality of reference genomic fragment sequences one by one in the present disclosure.

Optionally, referring to FIG. 6, the step 1021 includes following steps 10211 and 10212.

In step 10211, the reference genome sequence is segmented by a chromosome unit to obtain a plurality of reference chromosome genomic sequences.

In step 10212, each of the reference chromosome genomic sequences is segmented by a preset length to obtain the plurality of reference genomic fragment sequences.

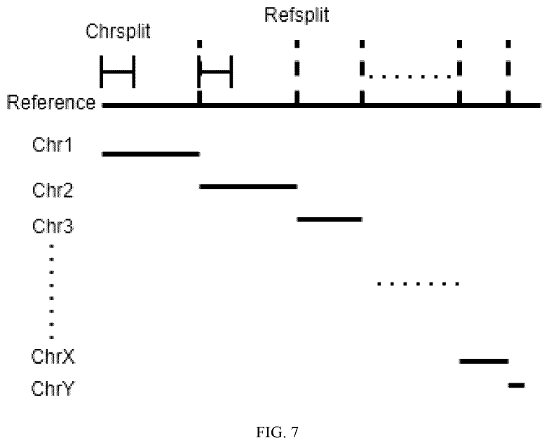

In some embodiments of the present disclosure, referring to FIG. 7, in which the reference genome sequence is firstly segmented with a chromosome as a segmenting unit. Refsplit indicates a split and obtained reference chromosome genomic sequence, and then the reference chromosome genomic sequence is further segmented by a preset length, and Chrsplit indicates the segmented and obtained reference genomic fragment sequence.

Optionally, the reference genome sequence includes a first converted reference genome sequence and a second converted reference genome sequence, and the genome methylation sequencing sequence at least includes a first amplified methylated genomic sequence and a second amplified methylated genomic sequence.

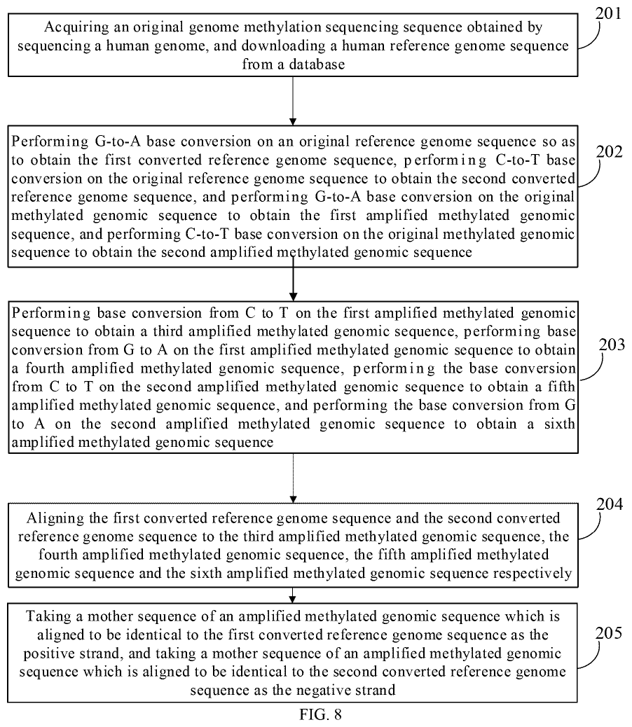

Referring to FIG. 8, the step 1022 includes following steps 201 and 205.

In the step 201, an original genome methylation sequencing sequence obtained by sequencing a human genome is acquired, and a human reference genome sequence is downloaded from a database.

In the step 202, C-to-T base conversion is performed on an original reference genome sequence so as to obtain the first converted reference genome sequence, G-to-A base conversion is performed on the original reference genome sequence to obtain the second converted reference genome sequence.

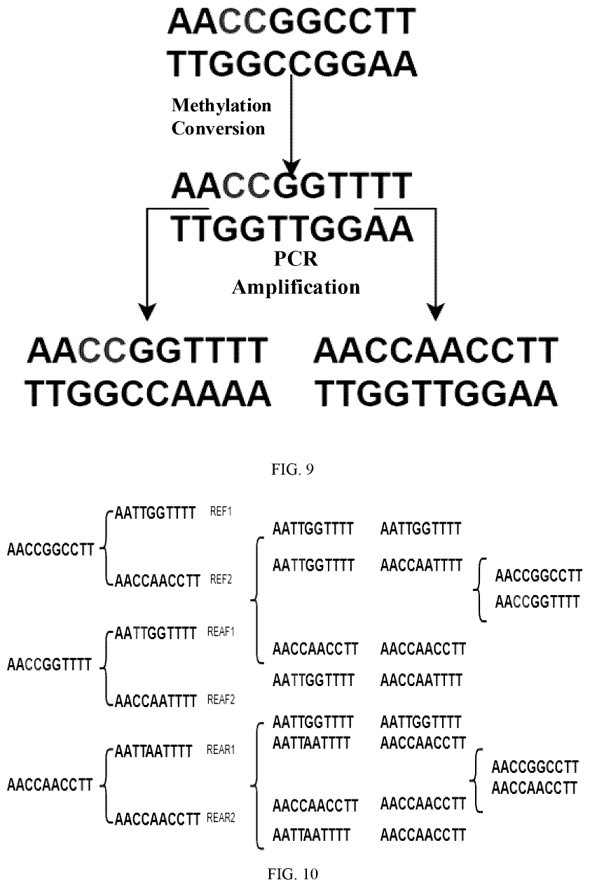

In the embodiment of the present disclosure, G represents guanine, A represents adenine, C represents cytosine, and T represents thymine. Since four possible sequences may be generated when PCR amplification is performed on the methylated conversion DNA in the upstream experiment, with reference to FIG. 9, it may be found that a resulting sequence may be regarded as a complementary pairing of a positive strand before unmethylated conversion, for which G is converted to A and C is converted to T respectively.

In the step 203, base conversion from C to T is performed on the first amplified methylated genomic sequence to obtain a third amplified methylated genomic sequence, base conversion from G to A is performed on the first amplified methylated genomic sequence to obtain a fourth amplified methylated genomic sequence, the base conversion from C to T is performed on the second amplified methylated genomic sequence to obtain a fifth amplified methylated genomic sequence, and the base conversion from G to A is performed on the second amplified methylated genomic sequence to obtain a sixth amplified methylated genomic sequence.

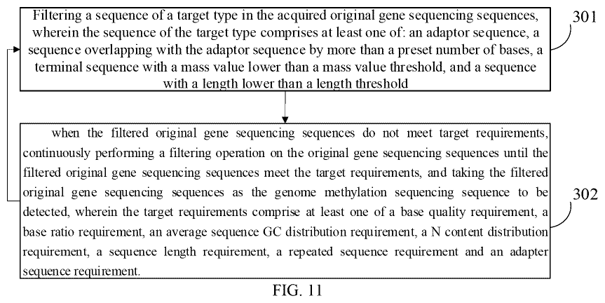

In the embodiment of the present disclosure, referring to FIG. 10, in order to distinguish positive and negative strands of the methylated sequencing sequence after methylation, C to T and G to A conversions are performed on the strand 1 (the first amplified methylated genomic sequence), which is then named REAF1 (the third amplified methylated genomic sequence) and REAF2 (the fourth amplified methylated genomic sequence), and the same operation is performed on the strand 2 (the second amplified methylated genomic sequence) so as to obtain REAR1 (the fifth amplified methylated genomic sequence) and REAR2 (the sixth amplified methylated genomic sequence). Meanwhile, the reference genome sequence subjected to C-to-T conversion is named REF1 (the first converted reference genome sequence), and the reference genome sequence subjected to G-to-A conversion is named REF2 (the second converted reference genome sequence).

In the step 204, the first converted reference genome sequence and the second converted reference genome sequence are aligned to the third amplified methylated genomic sequence, the fourth amplified methylated genomic sequence, the fifth amplified methylated genomic sequence and the sixth amplified methylated genomic sequence respectively.

In the embodiment of the present disclosure, also referring to FIG. 10, REF1 and REF2 are aligned to REAF1 and REAF2, REAR1 and REAR2 in a 2×2 alignment manner.

In step 205, a mother sequence of an amplified methylated genomic sequence which is aligned to be identical to the first converted reference genome sequence is taken as the positive strand, and a mother sequence of an amplified methylated genomic sequence which is aligned to be identical to the second converted reference genome sequence is taken as the negative strand.

In the embodiment of the present disclosure, referring to FIG. 10, when the aligned sequences are the same, that is, REF1 is identical to REAF1, a mother sequence of REAF1 is determined as the positive strand; and REF2 is identical to REAR2, and a mother sequence of REAR2 is determined as the negative strand.

Optionally, referring to FIG. 11, after the step 101, the method further includes following steps 301 and 302.

In the step 301, a sequence of a target type in the acquired original gene sequencing sequences is pruned. The sequence of the target type includes at least one of an adapter sequence, a sequence overlapping with the adapter sequence by more than a preset number of bases, a terminal sequence with a mass value lower than a mass value threshold, after completely pruning, and a sequence with a length lower than a length threshold is discarded to obtain pruned original gene sequencing sequences, and then the pruned original gene sequencing sequences are filtered.

In some embodiments of the present disclosure, possible adapter sequences are detected and removed through data filtering, and when a sequence is with overlapping bases between the sequences at both ends of reads and an adapter are greater than or equal to a preset base number, for example, 3, it is also regarded as the adapter sequence so as to be removed, and after the adapter is removed, a terminal of the sequence whose quality value is lower than a quality threshold, for example, 20 is pruned, and a fragment sequence whose length is less than, for example, 20 due to pruning is discarded, with a maximum allowable error rate of 0.1 (which is a number of errors divided by a length of a matching region). Of course, specific threshold parameters may be set according to the actual needs, which are not limited herein.

Further, a fastq format automatic detection script written in Perl languages may be used to identify whether a fastq file is of a Phred33 or Phred64 format (a text-based file format for storing biological sequences and corresponding base (or amino acid) quality) for the original gene sequencing sequences in advance, so as to ensure that formats of the original gene sequencing sequences meet a filtering standard.

In the step 302, when the pruned original gene sequencing sequences do not meet target requirements, filtering is continuously performed on the pruned original sequencing sequences until the filtered pruned-original sequencing sequences meet the target requirements, and then the filtered pruned-original sequencing sequences are taken as the genome methylation sequencing sequence to be detected. The target requirements include at least one of a base quality requirement, a base ratio requirement, an average sequence GC distribution requirement, a N content distribution requirement, a sequence length requirement, a repeated sequence requirement and an adapter sequence requirement.

In the embodiment of the present disclosure, data evaluation is performed on single-ended or double-ended sequencing data in terms of base quality, a base ratio, an average sequence GC distribution, a N content distribution, a sequence length, a repeated sequence and an adapter sequence, etc., and data quality evaluation is performed on the data-filtered sequencing data again according to a standard of the target requirements, so as to observe whether some unqualified indicators have been corrected, so as to ensure quality of sequencing sequences participating in subsequent identification.

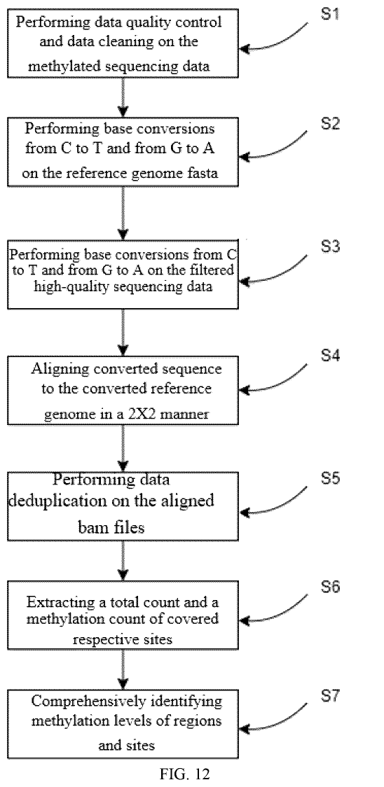

Optionally, FIG. 12 shows a flow chart of yet another method for analyzing genome methylation sequencing data according to some embodiments of the present disclosure.

S1 involves a data quality control and a data filtering module.

In the embodiment of the present disclosure, a number of reads and a number of bases before and after data quality control and data filtering of the methylated sequencing data are as follows in Table 1.

TABLE 1

| Name | Number of reads | Number of bases |
|---|---|---|
| Sample_fq1 | 183,772,579 | 18,193,485,321 |
| Sample_fq2 | 183,772,579 | 18,561,030,479 |
| Sample_fq1_qc | 175,403,025 | 16,294,145,923 |
| Sample_fq2_qc | 175,403,025 | 17,055,175,239 |

in which Sample_fq1 and Sample_fq2 are file names of double-ended sequencing original sequences, and Sample_fq1_qc and Sample_fq2_qc are sequence file names of Sample_fq1 and Sample_fq2 subjected to data quality control and filtering, respectively.

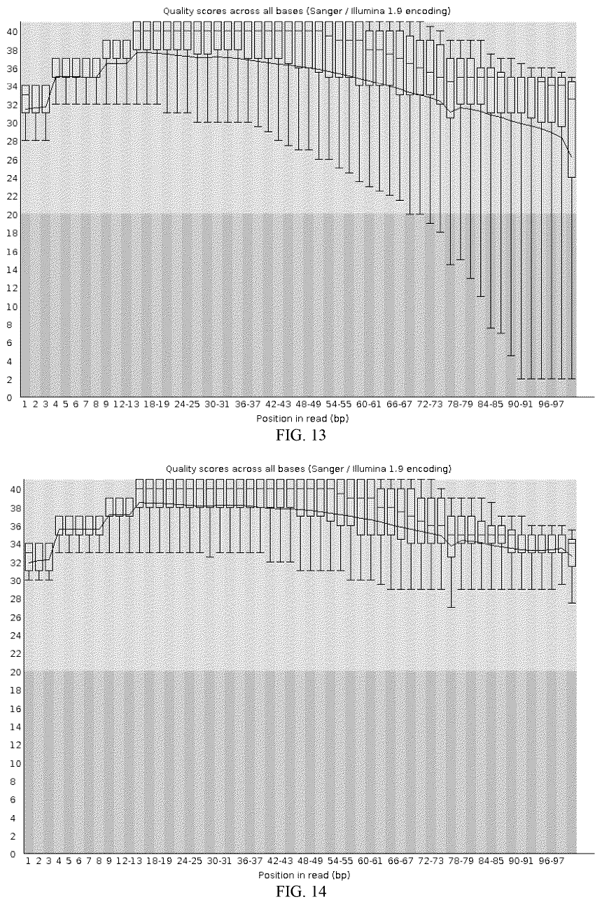

The base mass value distribution counted by reads positions before and after data quality control and data filtering may be referred to FIG. 13 and FIG. 14. It may be seen that before data quality control, last 30 base masses at the reads positions were relatively low, as shown in FIG. 13; and after data quality control, the base mass distribution is counted by the reads positions and mass values are substantially above 28, as shown in FIG. 14.

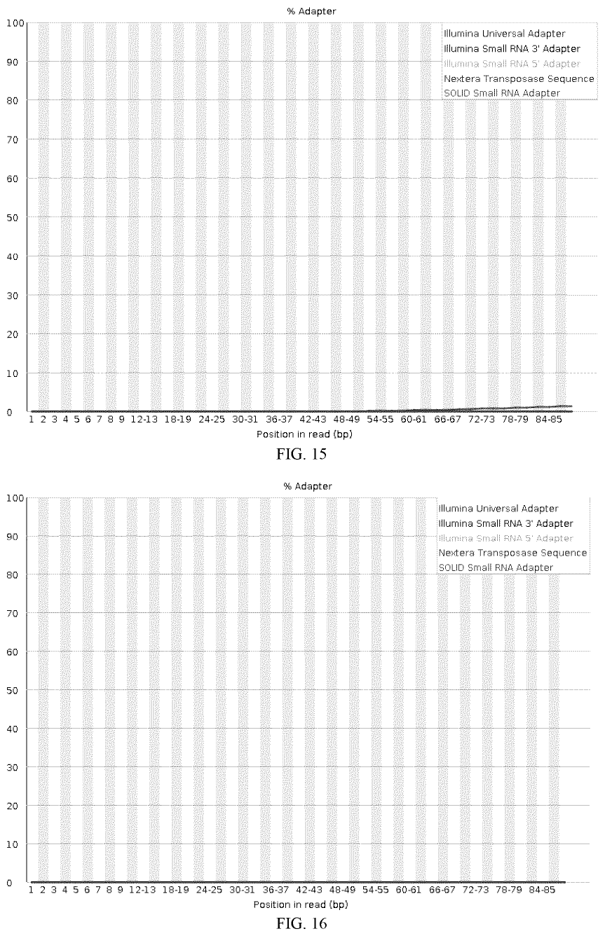

An adapter distribution counted by the reads positions before and after data quality control and data filtering may be seen in FIGS. 15 and 16. It may be seen that before the data quality control, some adapter sequences existed in last 20 bases at the reads positions, which is shown in FIG. 15; and after the data quality control, the adapter distribution is counted by the reads positions, and basically no adapter sequences existed, which is shown in FIG. 16.

In S2, sequence conversion is performed on the reference genome.

In the embodiment of the present disclosure, an original reference sequence is named genome.fa, the reference genome after conversion from G to A is named genome_mfa.GA_conversion.fa, and the reference genome after conversion from C to T is named genome_mfa.CT_conversion.fa.

In S3, sequence conversion is performed on the sequencing data subjected to the data quality control and the filtering.

In the embodiment of the present disclosure, names of the sequencing data subjected to the data quality control and filtering are Sample_fq1_qc.fastq and Sample_fq2_qc.fastq. After conversions from C to T and from G to A, Sample_fq1_qc.fastq forms two new sequence files, namely Sample_fq1_qc.CT_conversion.fastq and Sample_fq1_qc.GA_conversion.fastq; and Sample_fq2_qc.fastq forms two new sequence files, namely Sample_fq2_qc.CT_conversion.fastq and Sample_fq2_qc.GA_conversion.fastq.

In S4, the converted sequencing data is aligning to the converted reference genome.

In the embodiment of the present disclosure, the reference genome is firstly segmented by chromosome with a Refsplit width, and then the data is aligned in the previously introduced 2×2 method after segmentation within the chromosome with a specific Chrsplit width is completed.

In S5, aligned bam files are de-duplicated.

In the embodiment of the present disclosure, a number of paired reads before deduplication is 134701491, the bam files are sorted by coordinate and the same marked sequences are deduplicated, with a total repeated sequence of 1701997, accounting for 1.26%, and a number of paired reads after deduplication is 132999494.

In S6, a total count and a methylation count for respective sites after alignment are extracted.

In the embodiment of the present disclosure, taking a number 1 chromosome region from 9000 to 30000 as an example, part of extracted information is shown in Table 2 below (20 sites are randomly sampled):

TABLE 2

| Chromosome | Starting | Ending | Methylated | Unmethylated |
|---|---|---|---|---|
| Number | site | site | Count | Count |
| chr1 | 29337 | 29337 | 0 | 2 |
| chr1 | 29310 | 29310 | 0 | 2 |
| chr1 | 15883 | 15883 | 1 | 0 |
| chr1 | 14349 | 14349 | 2 | 1 |
| chr1 | 29144 | 29144 | 0 | 1 |
| chr1 | 20387 | 20387 | 8 | 1 |
| chr1 | 13756 | 13756 | 1 | 0 |
| chr1 | 10702 | 10702 | 2 | 1 |
| chr1 | 20156 | 20156 | 38 | 3 |
| chr1 | 10747 | 10747 | 1 | 0 |
| chr1 | 19683 | 19683 | 10 | 0 |
| chr1 | 29268 | 29268 | 0 | 7 |
| chr1 | 10708 | 10708 | 3 | 0 |
| chr1 | 29368 | 29368 | 0 | 12 |
| chr1 | 20254 | 20254 | 3 | 0 |
| chr1 | 14436 | 14436 | 0 | 1 |
| chr1 | 16070 | 16070 | 16 | 2 |
| chr1 | 29428 | 29428 | 0 | 1 |
| chr1 | 15090 | 15090 | 26 | 3 |
| chr1 | 29311 | 29311 | 0 | 13 |

In S7, methylation levels of regions and sites are identified.

In the embodiment of the present disclosure, the regional methylation level is identified according to the previously introduced method, taking the number 1 chromosome region from 9000 to 30000 as an example, wherein a Window width is set to be 2000 and a Step width is set to be 1000, intersection between the window obtained after sliding and the methylation information obtained in S6 is taken, and the obtained original methylation data information of part of windows is as follows in Table 3 (20 sites in a middle of a data set):

TABLE 3

| Chromosome | Starting | Ending | Methylated | Unmethylated | Chromosome | Starting site | Ending site |
|---|---|---|---|---|---|---|---|
| Number 1 | site | site | Count | Count | Number 2 | of window | of window |
| chr1 | 13079 | 13079 | 7 | 4 | chr1 | 12000 | 14000 |
| chr1 | 13079 | 13079 | 7 | 4 | chr1 | 13000 | 15000 |
| chr1 | 13216 | 13216 | 6 | 4 | chr1 | 12000 | 14000 |
| chr1 | 13216 | 13216 | 6 | 4 | chr1 | 13000 | 15000 |
| chr1 | 13217 | 13217 | 3 | 2 | chr1 | 12000 | 14000 |
| chr1 | 13217 | 13217 | 3 | 2 | chr1 | 13000 | 15000 |
| chr1 | 13283 | 13283 | 9 | 2 | chr1 | 12000 | 14000 |
| chr1 | 13283 | 13283 | 9 | 2 | chr1 | 13000 | 15000 |
| chr1 | 13284 | 13284 | 10 | 1 | chr1 | 12000 | 14000 |
| chr1 | 13284 | 13284 | 10 | 1 | chr1 | 13000 | 15000 |
| chr1 | 13302 | 13302 | 9 | 1 | chr1 | 12000 | 14000 |
| chr1 | 13302 | 13302 | 9 | 1 | chr1 | 13000 | 15000 |
| chr1 | 13303 | 13303 | 8 | 3 | chr1 | 12000 | 14000 |
| chr1 | 13303 | 13303 | 8 | 3 | chr1 | 13000 | 15000 |
| chr1 | 13418 | 13418 | 2 | 26 | chr1 | 12000 | 14000 |
| chr1 | 13418 | 13418 | 2 | 26 | chr1 | 13000 | 15000 |
| chr1 | 13504 | 13504 | 19 | 15 | chr1 | 12000 | 14000 |
| chr1 | 13504 | 13504 | 19 | 15 | chr1 | 13000 | 15000 |
| chr1 | 13529 | 13529 | 12 | 14 | chr1 | 12000 | 14000 |
| chr1 | 13529 | 13529 | 12 | 14 | chr1 | 13000 | 15000 |

Subsequently, a methylation level value Mbi of each window is calculated by formula (1) as shown in Table 4 below:

TABLE 4

| Chromosome | Starting site | Ending site | Methylated | Unmethylated | Methylation |
|---|---|---|---|---|---|
| Number 2 | of window | of window | Count of window | Count of window | level of window |
| chr1 | 9000 | 11000 | 91 | 7 | 0.928571429 |
| chr1 | 10000 | 12000 | 91 | 7 | 0.928571429 |
| chr1 | 11000 | 13000 | 1 | 0 | 1 |
| chr1 | 12000 | 14000 | 100 | 87 | 0.534759358 |
| chr1 | 13000 | 15000 | 625 | 144 | 0.812743823 |
| chr1 | 14000 | 16000 | 1157 | 130 | 0.898989899 |
| chr1 | 15000 | 17000 | 800 | 93 | 0.895856663 |
| chr1 | 16000 | 18000 | 612 | 58 | 0.913432836 |
| chr1 | 17000 | 19000 | 452 | 39 | 0.920570265 |
| chr1 | 18000 | 20000 | 313 | 35 | 0.899425287 |
| chr1 | 19000 | 21000 | 568 | 78 | 0.879256966 |
| chr1 | 20000 | 22000 | 264 | 44 | 0.857142857 |
| chr1 | 28000 | 30000 | 1 | 416 | 0.002398082 |
| chr1 | 29000 | 30000 | 1 | 416 | 0.002398082 |

Finally, the methylation level for the number 1 chromosome region from 9000 to 30000 was calculated as 0.7481512 using formula (2).

Secondly, according to the previously introduced comprehensive evaluation method of site methylation level, the window-site methylation level value is calculated using formula (3), and a part of obtained information is shown in Table 5 below (20 sites are randomly sampled):

TABLE 5

| | | | Starting | Ending | Site methylation |
|---|---|---|---|---|---|
| Chromosome | Starting | Ending | site of | site of | level value |
| Number 1 | site | site | window | window | of window |
| chr1 | 29300 | 29300 | 29000 | 30000 | 0 |
| chr1 | 13538 | 13538 | 13000 | 15000 | 0.014304291 |
| chr1 | 13283 | 13283 | 12000 | 14000 | 0.048128342 |
| chr1 | 10689 | 10689 | 10000 | 12000 | 0.030612245 |
| chr1 | 15849 | 15849 | 14000 | 16000 | 0.004662005 |
| chr1 | 20044 | 20044 | 19000 | 21000 | 0.009287926 |
| chr1 | 29307 | 29307 | 29000 | 30000 | 0 |
| chr1 | 19772 | 19772 | 19000 | 21000 | 0.027863777 |
| chr1 | 15834 | 15834 | 14000 | 16000 | 0.004662005 |
| chr1 | 29390 | 29390 | 29000 | 30000 | 0 |
| chr1 | 13216 | 13216 | 13000 | 15000 | 0.007802341 |
| chr1 | 20387 | 20387 | 19000 | 21000 | 0.012383901 |
| chr1 | 14888 | 14888 | 13000 | 15000 | 0.049414824 |
| chr1 | 29216 | 29216 | 29000 | 30000 | 0 |
| chr1 | 17571 | 17571 | 16000 | 18000 | 0.02238806 |
| chr1 | 16822 | 16822 | 16000 | 18000 | 0.01641791 |
| chr1 | 29298 | 29298 | 28000 | 30000 | 0 |
| chr1 | 15644 | 15644 | 14000 | 16000 | 0.003885004 |
| chr1 | 15207 | 15207 | 15000 | 17000 | 0.011198208 |
| chr1 | 20234 | 20234 | 20000 | 22000 | 0.13961039 |

Subsequently, the site methylation level value was calculated using formula (4), and a part of obtained information is shown in Table 6 below (20 sites are randomly sampled).

TABLE 6

| | | | comprehensive |
|---|---|---|---|
| | | | evaluation |
| Chromosome | Starting | Ending | value of site |
| Number 1 | site | site | methylation |
| chr1 | 29485 | 29485 | 0 |
| chr1 | 29128 | 29128 | 0 |
| chr1 | 29141 | 29141 | 0 |
| chr1 | 15770 | 15770 | 0.000948411 |
| chr1 | 10579 | 10579 | 0.020408163 |
| chr1 | 29166 | 29166 | 0 |
| chr1 | 17715 | 17715 | 0.001764599 |
| chr1 | 15750 | 15750 | 0.000948411 |
| chr1 | 29463 | 29463 | 0 |
| chr1 | 29479 | 29479 | 0 |
| chr1 | 16082 | 16082 | 0.024817402 |
| chr1 | 13216 | 13216 | 0.019943951 |
| chr1 | 15086 | 15086 | 0.019916627 |
| chr1 | 29484 | 29484 | 0 |
| chr1 | 19590 | 19590 | 0.004421551 |
| chr1 | 14711 | 14711 | 0 |
| chr1 | 29416 | 29416 | 0 |
| chr1 | 29325 | 29325 | 0 |
| chr1 | 10728 | 10728 | 0.010204082 |
| chr1 | 14791 | 14791 | 0.047779991 |

Of course, specific embodiments described above are only illustrative, and their specific usage modes may be set according to actual needs, which is not limited herein.

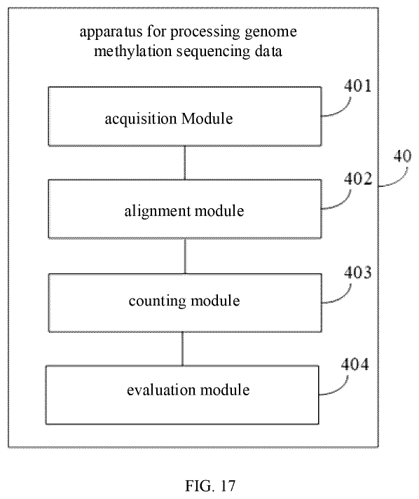

FIG. 17 schematically shows a schematic structural diagram of an apparatus 40 for analyzing genome methylation sequencing data according to the present disclosure, which includes an acquisition module 401, an alignment module 402, a counting module 403, an evaluation module 404,

The acquisition module 401 is configured to acquire a genome methylation sequencing sequence to be detected and a reference genome sequence.

The alignment module 402 is configured to align the genome methylation sequencing sequence to the reference genome sequence so as to obtain an alignment result.

The counting module 403 is configured to construct a window, and move the window from a first end of the alignment result to a second end of the alignment result successively, and count a methylation index of a part of the alignment result covered by the window at a different position during each movement, a step size of each movement of the window is smaller than a length of the window.

The evaluation module 404 is configured to analyze the counted methylation index of the part of the alignment result covered by a window at a respective different position, and output a comprehensive methylation evaluation result of the genome methylation sequencing sequence.

Optionally, when the comprehensive methylation evaluation result is the regional methylation level value of a target area in the alignment result, the methylation index includes: a total number of methylated bases at methylation sites for the alignment result covered by the window in the target area, and a total number of bases at the methylation sites;

The evaluation module 404 is further configured to:
calculate a regional window methylation level value corresponding to each window at the different position in the target area, according to a ratio of the total number of the methylated bases at the methylation sites to the total number of bases at the methylation sites included in the alignment result covered by a window at a respective different position in the target area; and
analyze the regional window methylation level value corresponding to each window at the different position in the target region to obtain the regional methylation level value of the target region in the genome methylation sequencing sequence.

Optionally, the evaluation module 404 is further configured to:
perform genome methylation sequencing and average the regional window methylation level value corresponding to each window at the different position in the target region, and calculate the regional methylation level value of the target region in the genome methylation sequencing sequence according to an average of the regional window methylation level value.

Optionally, when the comprehensive methylation evaluation result is a site methylation level value of a target site, the methylation index includes a total number of bases at the methylation sites in the alignment result covered by a window covering the target site and a number of methylated bases at the target site.

The evaluation module 404 is further configured to:
calculate a site-window methylation level value corresponding to a window covering a respective different position of the target site according to a ratio of the number of the methylated bases at the target site to the total number of the bases at the methylation sites in the alignment result covered by different windows covering the target site; and
analyze the site-window methylation level value corresponding to the window covering the respective different position of the target site to obtain the site methylation level value of the target site.

Optionally, the evaluation module 404 is further configured to:
perform genome methylation sequencing and average the site-window methylation level value corresponding to the window covering the respective different position of the target site, and calculate the site methylation level value of the target site in the genome methylation sequencing sequence according to an average of the site-window methylation level value.

Optionally, the counting module 403 is further configured to:
slide the window from the first end to the second end of the alignment result by a preset length, count a methylation index of the covered alignment result before a first sliding, and count a methylation index of the alignment result covered by the window after each sliding. A step size of each moving of the window is smaller than the length of the window.

Optionally, the alignment module 402 is further configured to:
segment the reference genome sequence to obtain a plurality of reference genomic fragment sequences; and
align each of the reference genomic fragment sequences to the genome methylation sequencing sequence in terms of negative and positive strands so as to obtain the alignment result.

Optionally, the alignment module 402 is further configured to:
segment the reference genome sequence by a chromosome unit to obtain a plurality of reference chromosome genomic sequences; and
segment each of the reference chromosome genomic sequences by a preset length to obtain the plurality of reference genomic fragment sequences.

Optionally, the reference genome sequence includes a first converted reference genome sequence and a second converted reference genome sequence, and the genome methylation sequencing sequence at least includes a first amplified methylated genomic sequence and a second amplified methylated genomic sequence.

The alignment module 402 is further configured to:
aligning each of the reference genomic fragment sequences to the genome methylation sequencing sequence in terms of negative and positive strands so as to obtain the alignment result, which includes:
performing base conversion on the first amplified methylated genomic sequence to at least obtain a third amplified methylated genomic sequence and obtain a fourth amplified methylated genomic sequence, performing base conversion on the second amplified methylated genomic sequence to at least obtain a fifth amplified methylated genomic sequence and a sixth amplified methylated genomic sequence;
aligning the first converted reference genome sequence and the second converted reference genome sequence to the third amplified methylated genomic sequence, the fourth amplified methylated genomic sequence, the fifth amplified methylated genomic sequence and the sixth amplified methylated genomic sequence respectively; and
taking a mother sequence of an amplified methylated genomic sequence which is aligned to be identical to the first converted reference genome sequence as the positive strand, and taking a mother sequence of an amplified methylated genomic sequence which is aligned to be identical to the second converted reference genome sequence as the negative strand.

Optionally, the alignment module 402 is further configured to:
perform base conversion from C to T on the first amplified methylated genomic sequence to obtain a third amplified methylated genomic sequence, perform base conversion from G to A on the first amplified methylated genomic sequence to obtain a fourth amplified methylated genomic sequence; and
perform the base conversion from C to T on the second amplified methylated genomic sequence to obtain a fifth amplified methylated genomic sequence, and perform the base conversion from G to A on the second amplified methylated genomic sequence to obtain a sixth amplified methylated genomic sequence.

Optionally, the acquisition module 401 is further configured to:
prune a sequence of a target type in the acquired original gene sequencing sequences, wherein the sequence of the target type includes at least one of: an adapter sequence, a sequence overlapping with the adapter sequence by more than a preset number of bases, a terminal sequence with a mass value lower than a mass value threshold, and after completely pruning, discard a sequence with a length lower than a length threshold to obtain pruned original gene sequencing sequences, and then filter the pruned original gene sequencing sequences; and
when the pruned original sequencing sequences do not meet target requirements, continuously perform filtering on the pruned original sequencing sequences until the filtered pruned-original sequencing sequences meet the target requirements, and then take the filtered pruned-original sequencing sequences as the genome methylation sequencing sequence to be detected, wherein the target requirements include at least one of a base quality requirement, a base ratio requirement, an average sequence GC distribution requirement, a N content distribution requirement, a sequence length requirement, a repeated sequence requirement and an adapter sequence requirement.

According to the embodiment of the present disclosure, the comprehensive methylation evaluation result obtained by integrating counted methylation indexes for different windows with multiple coverage can summarize methylation indexes of the comparison result covered by multiple windows covering specific areas or characteristic sites, so that influence of local errors in the sequencing sequence on accuracy of the methylation evaluation result of a whole area or site is reduced as much as possible, and the accuracy of the methylation evaluation result of the methylation sequencing sequence may be improved.

The embodiments of each component in the present disclosure may be implemented by hardware, or by software modules running on one or more processors, or by their combination. A person skilled in the art should understand that the microprocessor or digital signal processor (DSP) may be used in practice to realize some or all functions of some or all components in the calculation and processing equipment according to the embodiments of the present disclosure the present disclosure. The present disclosure may also be implemented as the equipment or device programs (for example, computer programs and computer program products) used to execute part or all of the methods described here. The programs of implementing the present disclosure may be stored in a computer-readable medium, or can have the form of one or more signals. Such signals may be downloaded from the Internet site, or provided on the carrier signal, or provided in any other form.

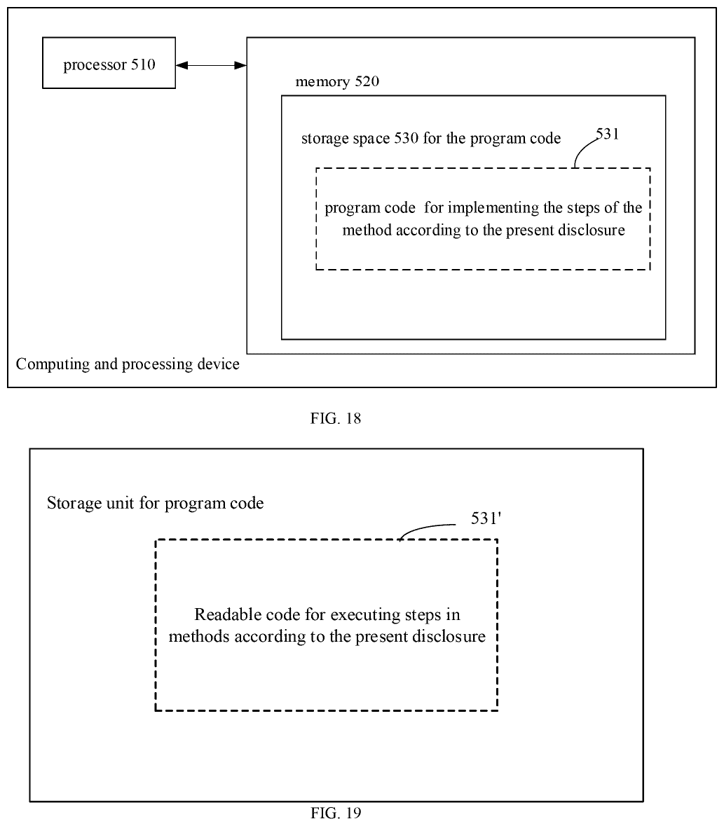

For example, FIG. 18 shows a calculating and processing device that can implement the method according to the present disclosure. The calculating and processing device traditionally includes a processor 510 and a computer program product or computer-readable medium in the form of a memory 520. The memory 520 may be electronic memories such as flash memory, EEPROM (Electrically Erasable Programmable Read Only Memory), EPROM, hard disk or ROM. The memory 520 has the storage space 530 of the program code 531 for implementing any steps of the above method. For example, the storage space 530 for program code may contain program codes 531 for individually implementing each of the steps of the above method. Those program codes may be read from one or more computer program products or be written into the one or more computer program products. Those computer program products include program code carriers such as a hard disk, a compact disk (CD), a memory card or a floppy disk. Such computer program products are usually portable or fixed storage units as shown in FIG. 19. The storage unit may have storage segments or storage spaces with similar arrangement to the memory 520 of the calculating and processing device in FIG. 18. The program codes may, for example, be compressed in a suitable form. Generally, the storage unit contains a computer-readable code 531′, which may be read by a processor like 510. When those codes are executed by the calculating and processing device, the codes cause the calculating and processing device to implement each of the steps of the method described above.

It should be understood that although the steps in the flow chart of the figures are displayed in turn according to the instructions of the arrows, these steps are not necessarily performed in the order indicated by the arrows. Unless this article makes it clear that there are no strict order restrictions on the execution of these steps, they may be executed in other order. Moreover, at least part of the steps in the flow chart can include multiple sub-steps or multiple stages. These sub-steps or stages are not necessarily completed at the same time, but may be executed at different times. The order of execution is not necessarily sequential, but may be performed by taking turns or alternately with at least part of sub-steps of other steps or stages of other steps.

Reference herein to “one embodiment,” “an embodiment,” or “one or more embodiments” means that a particular feature, structure, or characteristic described in connection with an embodiment is included in at least one embodiment of the present disclosure. Also, please note that instances of the phrase “in one embodiment” herein are not necessarily all referring to the same embodiment.

In the specification provided herein, numerous specific details are set forth. It will be understood, however, that the embodiments of the present disclosure may be practiced without these specific details. In some instances, well-known methods, structures, and techniques have not been shown in detail in order not to obscure an understanding of this specification.

In the claims, any reference signs between parentheses should not be construed as limiting the claims. The word “include” does not exclude elements or steps that are not listed in the claims. The word “a” or “an” preceding an element does not exclude the existing of a plurality of such elements. The present application may be implemented by means of hardware including several different elements and by means of a properly programmed computer. In unit claims that list several devices, some of those devices may be embodied by the same item of hardware. The words first, second, third and so on do not denote any order. Those words may be interpreted as names.

Finally, it should be noted that the above embodiments are only intended to illustrate technical schemes of the present disclosure, but not to limit it. Although the present disclosure has been described in detail with reference to the foregoing embodiments, it should be understood by ordinary skilled in the art that modifications may be made to the technical schemes described in the foregoing embodiments, or equivalent substitutions may be made to some technical features thereof. These modifications or substitutions do not make essence of corresponding technical schemes depart from the spirit and scope of the technical schemes of the embodiments of the present disclosure.

1. A method for analyzing genome methylation sequencing data, comprising:
acquiring a genome methylation sequencing sequence to be detected and a reference genome sequence;
aligning the genome methylation sequencing sequence to the reference genome sequence so as to obtain an alignment result;
constructing a window, and moving the window from a first end of the alignment result to a second end of the alignment result successively, and counting a methylation index of a part of the alignment result covered by the window at a different position during each movement, a step size of each movement of the window being smaller than a length of the window; and
analyzing a counted methylation index of the part of the alignment result covered by a window at a respective different position, and outputting a comprehensive methylation evaluation result of the genome methylation sequencing sequence.

2. The method according to claim 1, wherein when the comprehensive methylation evaluation result is the regional methylation level value of a target area in the alignment result, the methylation index comprising a total number of methylated bases at methylation sites for the alignment result covered by the window in the target area, and a total number of bases at the methylation sites;
the step of analyzing the counted methylation index of the part of the alignment result covered by the window at the respective different position and outputting the comprehensive methylation evaluation result of the genome methylation sequencing sequence comprises:
calculating a regional window methylation level value corresponding to each window at the different position in the target area, according to a ratio of the total number of the methylated bases at the methylation sites to the total number of bases at the methylation sites included in the alignment result covered by a window at a respective different position in the target area; and
analyzing the regional window methylation level value corresponding to each window at the different position in the target region to obtain the regional methylation level value of the target region in the genome methylation sequencing sequence.

3. The method according to claim 2, wherein analyzing the regional window methylation level value corresponding to each window at the different position in the target region to obtain the regional methylation level value of the target region in the genome methylation sequencing sequence comprises:
averaging the regional window methylation level value corresponding to each window at the different position in the target region, and calculating the regional methylation level value of the target region in the genome methylation sequencing sequence according to an average of the regional window methylation level value.

4. The method according to claim 1, wherein when the comprehensive methylation evaluation result is a site methylation level value of a target site, the methylation index comprises a total number of bases at the methylation sites in the alignment result covered by a window covering the target site and a number of methylated bases at the target site;
the step of analyzing the counted methylation index of the part of the alignment result covered by a window at a respective different position and outputting a comprehensive methylation evaluation result of the genome methylation sequencing sequence comprises:
calculating a site-window methylation level value corresponding to a window covering a respective different position of the target site according to a ratio of the number of the methylated bases at the target site to the total number of the bases at the methylation sites in the alignment result covered by different windows covering the target site; and
analyzing the site-window methylation level value corresponding to the window covering the respective different position of the target site to obtain the site methylation level value of the target site.

5. The method according to claim 4, wherein analyzing the site-window methylation level value corresponding to the window covering the respective different position of the target site to obtain the site methylation level value of the target site comprises:
averaging the site-window methylation level value corresponding to the window covering the respective different position of the target site, and calculating the site methylation level value of the target site in the genome methylation sequencing sequence according to an average of the site-window methylation level value.

6. The method according to claim 1, wherein sliding the window from the first end of the alignment result to the second end of the alignment result successively and counting the methylation index of the part of the alignment result covered by the window at the different position during each movement comprises:
sliding the window from the first end of the alignment result to the second end of the alignment result by a preset length, counting a methylation index of a covered alignment result before a first sliding, and counting a methylation index of the alignment result covered by the window after each sliding, wherein a step size of each moving of the window is smaller than the length of the window.

7. The method according to claim 1, wherein aligning the genome methylation sequencing sequence to the reference genome sequence so as to obtain the alignment result comprises:
segmenting the reference genome sequence to obtain a plurality of reference genomic fragment sequences; and
aligning each of the reference genomic fragment sequences to the genome methylation sequencing sequence in terms of negative and positive strands so as to obtain the alignment result.

8. The method according to claim 7, wherein segmenting the reference genome sequence to obtain the plurality of reference genomic fragment sequences comprises:
segmenting the reference genome sequence by a chromosome unit to obtain a plurality of reference chromosome genomic sequences; and
segmenting each of the reference chromosome genomic sequences by a preset length to obtain the plurality of reference genomic fragment sequences.

9. The method according to claim 7, wherein the reference genome sequence comprises a first converted reference genome sequence and a second converted reference genome sequence, and the genome methylation sequencing sequence at least comprises a first amplified methylated genomic sequence and a second amplified methylated genomic sequence;
the step of aligning each of the reference genomic fragment sequences to the genome methylation sequencing sequence in terms of negative and positive strands so as to obtain the alignment result comprises:
performing base conversion on the first amplified methylated genomic sequence to at least obtain a third amplified methylated genomic sequence and obtain a fourth amplified methylated genomic sequence, performing base conversion on the second amplified methylated genomic sequence to at least obtain a fifth amplified methylated genomic sequence and a sixth amplified methylated genomic sequence;
aligning the first converted reference genome sequence and the second converted reference genome sequence to the third amplified methylated genomic sequence, the fourth amplified methylated genomic sequence, the fifth amplified methylated genomic sequence and the sixth amplified methylated genomic sequence respectively; and
taking a mother sequence of an amplified methylated genomic sequence which is aligned to be identical to the first converted reference genome sequence as the positive strand, and taking a mother sequence of an amplified methylated genomic sequence which is aligned to be identical to the second converted reference genome sequence as the negative strand.

10. The method according to claim 9, wherein the step of performing base conversion on the first amplified methylated genomic sequence to at least obtain the third amplified methylated genomic sequence and obtain the fourth amplified methylated genomic sequence, and performing base conversion on the second amplified methylated genomic sequence to at least obtain the fifth amplified methylated genomic sequence and the sixth amplified methylated genomic sequence comprises:
performing base conversion from C to T on the first amplified methylated genomic sequence to obtain the third amplified methylated genomic sequence, performing base conversion from G to A on the first amplified methylated genomic sequence to obtain the fourth amplified methylated genomic sequence; and
performing the base conversion from C to T on the second amplified methylated genomic sequence to obtain the fifth amplified methylated genomic sequence, and performing the base conversion from G to A on the second amplified methylated genomic sequence to obtain the sixth amplified methylated genomic sequence.

11. The method according to claim 1, wherein after methylated genomic sequencing to acquire the genome methylation sequencing sequence to be detected and obtaining the reference genome sequence by downloading from a database, the method further comprises:
pruning a sequence of a target type in the acquired original gene sequencing sequences, wherein the sequence of the target type comprises at least one of: an adapter sequence, a sequence overlapping with the adapter sequence by more than a preset number of bases, a terminal sequence with a mass value lower than a mass value threshold, after completely pruning, discarding a sequence with a length lower than a length threshold to obtain pruned original gene sequencing sequences, and then filtering the pruned original gene sequencing sequences; and
when the pruned original gene sequencing sequences do not meet target requirements, continuously performing filtering on the pruned original gene sequencing sequences until filtered pruned-original gene sequencing sequences meet the target requirements, and taking the filtered pruned-original gene sequencing sequences as the genome methylation sequencing sequence to be detected, wherein the target requirements comprise at least one of a base quality requirement, a base ratio requirement, an average sequence GC distribution requirement, a N content distribution requirement, a sequence length requirement, a repeated sequence requirement and an adapter sequence requirement.

12. (canceled)

13. A computing and processing device, comprising:
a memory with a computer-readable code stored therein;
one or more processors, the computing and processing device executing the method for analyzing the genome methylation sequencing data according to claim 1 when the computer-readable code is executed by the one or more processors.

14. (canceled)

15. A non-transient computer-readable medium with a computer program of the method for analyzing the genome methylation sequencing data according to claim 1 stored therein.

16. The computing and processing device according to claim 13, wherein when the comprehensive methylation evaluation result is the regional methylation level value of a target area in the alignment result, the methylation index comprising a total number of methylated bases at methylation sites for the alignment result covered by the window in the target area, and a total number of bases at the methylation sites;
the operation of analyzing the counted methylation index of the part of the alignment result covered by the window at the respective different position and outputting the comprehensive methylation evaluation result of the genome methylation sequencing sequence comprises:
calculating a regional window methylation level value corresponding to each window at the different position in the target area, according to a ratio of the total number of the methylated bases at the methylation sites to the total number of bases at the methylation sites included in the alignment result covered by a window at a respective different position in the target area; and
analyzing the regional window methylation level value corresponding to each window at the different position in the target region to obtain the regional methylation level value of the target region in the genome methylation sequencing sequence.

17. The computing and processing device according to claim 16, wherein analyzing the regional window methylation level value corresponding to each window at the different position in the target region to obtain the regional methylation level value of the target region in the genome methylation sequencing sequence comprises:
averaging the regional window methylation level value corresponding to each window at the different position in the target region, and calculating the regional methylation level value of the target region in the genome methylation sequencing sequence according to an average of the regional window methylation level value.

18. The computing and processing device according to claim 13, wherein when the comprehensive methylation evaluation result is a site methylation level value of a target site, the methylation index comprises a total number of bases at the methylation sites in the alignment result covered by a window covering the target site and a number of methylated bases at the target site;
the operation of analyzing the counted methylation index of the part of the alignment result covered by a window at a respective different position and outputting a comprehensive methylation evaluation result of the genome methylation sequencing sequence comprises:
calculating a site-window methylation level value corresponding to a window covering a respective different position of the target site according to a ratio of the number of the methylated bases at the target site to the total number of the bases at the methylation sites in the alignment result covered by different windows covering the target site; and
analyzing the site-window methylation level value corresponding to the window covering the respective different position of the target site to obtain the site methylation level value of the target site.

19. The computing and processing device according to claim 18, wherein analyzing the site-window methylation level value corresponding to the window covering the respective different position of the target site to obtain the site methylation level value of the target site comprises:
averaging the site-window methylation level value corresponding to the window covering the respective different position of the target site, and calculating the site methylation level value of the target site in the genome methylation sequencing sequence according to an average of the site-window methylation level value.

20. The computing and processing device according to claim 13, wherein sliding the window from the first end of the alignment result to the second end of the alignment result successively and counting the methylation index of the part of the alignment result covered by the window at the different position during each movement comprises:
sliding the window from the first end of the alignment result to the second end of the alignment result by a preset length, counting a methylation index of a covered alignment result before a first sliding, and counting a methylation index of the alignment result covered by the window after each sliding, wherein a step size of each moving of the window is smaller than the length of the window.

21. The computing and processing device according to claim 13, wherein aligning the genome methylation sequencing sequence to the reference genome sequence so as to obtain the alignment result comprises:
segmenting the reference genome sequence to obtain a plurality of reference genomic fragment sequences; and
aligning each of the reference genomic fragment sequences to the genome methylation sequencing sequence in terms of negative and positive strands so as to obtain the alignment result.

22. The computing and processing device according to claim 21, wherein segmenting the reference genome sequence to obtain the plurality of reference genomic fragment sequences comprises:
segmenting the reference genome sequence by a chromosome unit to obtain a plurality of reference chromosome genomic sequences; and
segmenting each of the reference chromosome genomic sequences by a preset length to obtain the plurality of reference genomic fragment sequences.
